(19) United States
(12) Patent Application Publication
(10) Pub. No.: US 2020/0016173 A1
(43) Pub. Date: Jan. 16, 2020

(54) Use of Exemestane for the Treatment of Gastric Cancer

(71) Applicant: CHINA MEDICAL UNIVERSITY, Taichung City (TW)

(72) Inventor: Wen-Lung Ma, Taichung City (TW)

(21) Appl. No.: 16/328,501

(22) PCT Filed: Aug. 25, 2017
(86) PCT No.: PCT/CN2017/099180

§ 371 Date: Feb. 26, 2019

Related U.S. Application Data
(60) Provisional application No. 62/380,415, filed on Aug. 27, 2016.

(51) Int. Cl. A61K 31/5685; A61P 35/00; A61K 31/513; A61K 31/17; A61K 31/704; A61K 31/407; A61K 33/243; A61K 31/337

(52) U.S. Cl. CPC A61K 31/5685; A61P 35/00; A61K 31/513; A61K 31/337; A61K 31/704; A61K 31/407; A61K 33/243; A61K 31/17

(57) ABSTRACT

Gastric cancer is treated with an aromatase irreversible steroidal inhibitor such as exemestane and derivatives thereof. The methods may further comprise the antecedent step of identifying the person as having gastric cancer and/or being in need of exemestane therapy, and/or the subsequent step of monitoring status of the gastric cancer or a biomarker thereof.

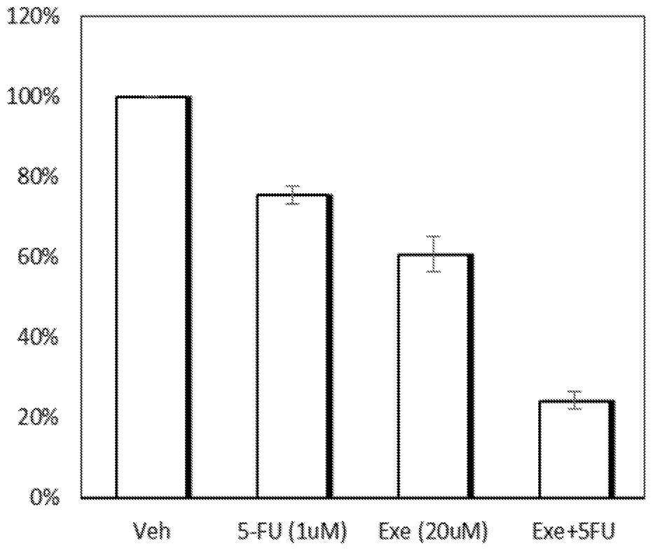

### INTRODUCTION

Gastric cancer (GCa) is the third leading cause of cancer death worldwide. The high mortality rates of GCa are due to late diagnosis [1] and a lack of effective adjuvant therapy agents [2]. The prognosis and survival is very poor for advanced stage cancer patients receiving gastrectomy, with only a 30% 5-year survival rate [3]. However, there is also limited effective chemotherapy for early stage cancer patients [4, 5]. One meta-analysis review of surgery and chemotherapy in GCa patients reported a limited efficacy of current standard therapy [6]. Therefore, it is of great importance to find a novel strategy for GCa therapy.

GCa patients are predominantly male [7]; however studies of gender factor involvement have been inconclusive. One epidemiological survey of female factors, e.g., reproductive age, ovariectomy surgery, breast-feeding, pregnancy, contraceptive agents, etc. suggested that an estrogenic signal suppresses the incidence of GCa [8-10], while a large-scale survey (1299 patients) indicates that female factors contribute to poor survival of GCa, and male factors contribute to GCa patient survival following surgery [11]. Additionally, Progesterone Receptor (PR) expression is significantly upregulated in GCa tissue [12], although serum progesterone levels do not correlated with incidences of GCa [13]. Moreover, serum testosterone levels are significantly decreased in incidences of recurring GCa [14], and low testosterone levels are correlated with post-surgery complications [15].

The initial biochemical process of steroidogenesis begins by converting cholesterol to pregnolone. Extracellular inflow is considered to be the major cellular cholesterol resource [16]. Lipoproteins, lipid carriers that engulf through the lipoprotein receptor, are one major route to provide cholesterol into cells. Some reports have suggested a connection between lipoprotein-cholesterol circulation and GCa: cholesterol-rich lipid droplet are commonly observed in GCa lesions; the lipoprotein receptor has been observed expressed in GCa or parental mucosa [17]; and the lipoprotein loading content might affect GCa disease development [18]. Among lipoproteins, low-density lipoproteins (LDL) and high-density lipoprotiens (HDL) are major cholesterol carriers in circulation, and HDL-C may be a risk factor in GCa [19].

To analyze the L/R route to steroidogenesis pathway in patients, utilized a web-based survival analyzer (Kaplan-Meier plotter) to test candidate genes in GCa disease survival and calculate the importance of gene clusters in GCa patients of unmet medical needs. The strategy uses meta-analysis of online cDNA microarray databases that predict the outcome in appropriately powered cohorts and provides a feasible, unbiased and genome-wide approach to analyze genes in cancer progression [20, 21]. We also disclose a novel GCa therapy based on CYP19A1 inhibition.

### SUMMARY OF THE INVENTION

The invention provides methods and compositions for treating gastric cancer with an aromatase irreversible steroidal inhibitor: exemestane (6-methylenandrosta-1,4-diene-3,17-di one).

In an aspect the invention provides a method of treating gastric cancer, comprising administering to a person in need thereof exemestane.

In embodiments:
the method further comprises the antecedent step of identifying the person as having gastric cancer and/or being in need of exemestane therapy;
the method further comprises the subsequent step of monitoring status of the gastric cancer or a biomarker thereof, such as androgen receptor (AR), progesterone receptor (PR), estrogen receptor 1; ERα (ESR1) and estrogen receptor 2; ERβ (ESR2);
the gastric cancer is HER2-negative;
the person is a gastrectomy patient; and/or
the person is contraindicated for gastrectomy.

In an aspect the invention provides use of exemestane in the manufacture of a medicament for treating gastric cancer.

In an aspect the invention provides a pharmaceutical composition formulated and/or labeled for treating gastric cancer comprising exemestane, and optionally a different gastric cancer drug, such as 5-FU (fluorouracil) or its analog capecitabine, BCNU (carmustine), methyl-CCNU (semustine), doxorubicin, mitomycin C, cisplatin and taxotere, preferably in unit dosage form.

The pharmaceutical compositions of the invention may be administered by any suitable route of administration, including orally, nasally, rectally, intravaginally, parenterally, intracisternally and topically, as by powders, ointments or drops, including buccally and sublingually. The preferred routes of administration are orally and parenterally.

The invention encompasses all combination of the particular embodiments recited herein, as if each combination had been laboriously recited.

### BRIEF DESCRIPTION OF THE DRAWING

FIGURE Exemestane and 5-fluouricil (5-FU) synergistically suppress gastric cancer cell growth

### DESCRIPTION OF PARTICULAR EMBODIMENTS OF THE INVENTION

The examples below are non-limiting and are merely representative of various aspects and features of the present invention.

Linking Lipoprotein/Receptor Route to Steroidogenesis in GCa Progression

The Kaplan Meier Survival Analyzer provides a platform to evaluate gene expression in GCa progression. The importance of sex steroid hormone nuclear receptor expression in 5-year OS of all GCa patients was weighted without differentiating between sexes. Four major nuclear receptors, including AR (androgen receptor), PR (progesterone receptor), ESR1 (estrogen receptor 1; ERα) and ESR2 (estrogen receptor 2; ERβ) are GCa progression promoter. The Hazard Ratios (HR) of each are: 1.42 (1.18-1.72; p=2.2e-04) for AR, 1.61 (1.3-1.99; p=1.4e-05) for PR, 1.56 (1.28-1.89; p=6.5e-06) for ESR1 and 1.58 (1.32-1.89; p=3.4e-07) for ESR2. Therefore, the four nuclear receptors are independent prognosis markers in GCa, regardless of gender or serum hormone levels.

To determine if the L/R route participates in GCa 5-year OS, LDLR, LRP6 (LDLR related protein 6) (26), SR-B1 (Scavenger receptor-B1, HDL receptor[22]) and LPL (lipoprotein lipase) were weighted in relation to 5-year OS. The HRs of each were: 1.23 (1.04-1.47; p=0.018) for LDLR, 2.1 (1.72-2.57; p=6.9e-14) for LRP6, 2 (1.61-2.48; p=1.5e-10), and 1.38 (1.16-1.65; p=3.8e-04) for LPL. This indicates that the L/R route shuttles cholesterol into GCa cells to facilitate cancer progression.

Since the PR is a GCa progression independent promoter and the L/R route elevates cellular cholesterol content to promote GCa, the steroidogenic enzyme toward progesterone in GCa 5-years OS was examined. The CYP11A1 (cholesterol to pregnolone), CYP17 (pregnolone to 17α-hydroxy-pregnolone and Dihydroxyepiandrostendiol (DHEA)), HSD3B1 (pregnolone to progesterone; 17α-hydroxy-pregnolone to 17α-hydroxy-progesterone; DHEA to androstenedione; and androstenediol to testosterone) and HSD17B1 (DHEA to androstenediol) are involved in the production of progesterone. Therefore, these four enzymes were adjusted and all were found to be GCa progression promoter. The HRs were 1.36 (1.14-1.64; p=8.9e-04) for CYP11A1, 1.47 (1.22-1.77; p=5.5e-05) for CYP17, 1.67 (1.4-1.99; p=9.3e-09) for HSD3B1 and 1.24 (1.04-1.48; p=0.014) for HSD17B1. These data indicate that progesterone production enzymes are GCa progression promoters. The pathological conversions of sex hormones from pregnolone to androstenediol and to testosterone are GCa progression favorable biochemical process.

The critical enzymes governing the conversion of androstenediol or testosterone to estradiol (active form of estrogen; CYP19A1) and testosterone to dihydrotestosterone (DHT, active form of androgen; SRD5A1) were examined. This was done to associate the ligand and receptor function in GCa. CYP19A1 is a poor GCa prognosis marker with HR=1.92 (1.57-2.34; p=1.1e-10); however, SDR5A1 is good GCa prognosis marker with HR=0.64 (0.54-0.77; p=1.3e-06). Thus, the pathological conversion of steroidogenesis in GCa is to favor progesterone and estradiol production, but not DHT.

Together, these data show that the L/R route shuttles cholesterol into tumors, which is linked to steroidogenesis enzymes and to producing ligands for PR and ESRs to promote GCa progression. Further, DHT anabolism is not a favorable pathological biochemical process to promote GCa progression.

Algorithm of HR Score Determined CYP19A1 could be Novel Target for GCa Therapy

To score gene clusters responsible for de novo synthesis of progesterone, estradiol, or DHT, we developed and utilized an algorithm (Formula 1) to test the importance of steroidogenic lipidomes in GCa.

HR Score=(Avg of HR gene sets)=Σ(HRn−1)X(−log₁₀(p value))/n×100

The HR of each gene is minus one, to adjust the effect of genes, multiplied with negative log 10 (p-value) to balance the importance of genes. The summed score is divided by the number of gene, and multiplied by 100 to get the HR score, or average HR of each gene. The threshold was 100 to indicate significance of gene clusters. HR scores >100 can be indicated as significant to be targeted, whereas HR scores ≤100 indicate a less value to be targeted for GCa therapy.

Current therapeutic regimens for GCa include surgery or chemotherapy [23]. Incomplete gastrectomy patients usually receive combined 5-fluouricil (5-FU) treatments as adjuvant chemotherapies [23, 24]. The median survival rate for patients undergoing surgery and 5-FU ranges from 36 to 91 months [24]. Anti-HER2 therapy has been introduced to HER2 positive (HER2+) GCa patients [25]. However, the anti-HER2 regimen exhibits only marginal survival benefits [25]. Therefore, understanding unmet medical needs of GCa requires evaluating patient subgroups that underwent surgery, surgery and 5-FU therapy and HER2 expression status. The KM plotter provides survival information for those subcategories of GCa patients.

CYP11A1 and HSD3A1 are responsible for the production of progesterone, as shown in the anabolic pathway for progesterone. The HR score for progesterone production is 54.02 in surgery, 9.19 in surgery and 5-FU, 259.85 in HER2 negative (HER2−), and 36.79 in HER2+ patients. The HR score in HER2− patients is high, which indicates targeting progesterone production might be effective in HER2−GCa patients. CYP11A1, CYP17, HSD17B1 and CYP19A1 are responsible for the production of estradiol. The HR score for estradiol production is 125.25 in surgery, 45.06 in surgery and 5-FU, 215.03 in HER2−, and 166.18 in HER2+ patients. Since the HR score in surgery, HER2+ and HER2− patients are high, targeting estradiol production might be effective. The anabolic pathway of DHT production shows that CYP11A1, CYP17, HSD17B1 and SRD5A1 are responsible for the production of DHT. The HR score for DHT production is 48.31 in surgery, 23.32 in surgery and 5-FU, 87.66 in HER2−, and 43.72 in HER2+ patients.

The analysis of steroidogenic lipidomes revealed that CYP11A1 and CYP19A1 are progression dominant genes in various categories of GCa patients. Furthermore, we implemented TCGA data to estimate their expressions in non-tumor (NT) versus tumor parental (TP) in GCa patients. It was seen that CYP11A1 is lower (p=0.019) but CYP19A1 is higher (p=0.008) in TP compared to their NT counterpart. In addition, the non-matched comparison also consistently found lower CYP11A1 (p=0.02) but higher CYP19A1 (p<0.0001) expressions in TP compared to the NT lesions. These data indicate that targeting CYP19A1 should have a better response in tumors compared to non-tumor gastric tissue. Finally, we weighted CYP19A1 expression to associate with another patient cohort from TCGA. The data clearly demonstrated that high CYP19A1 expression is linked to poor overall survival compared to in low expression.

Targeting CYP19A1 as Novel Gastric Cancer Therapy

In order to test that targeting CYP19A1 could be an effective therapy for GCa, three CYP19A1 inhibitor were applied on SNU1 and SC-M1 human GCa cell lines. The type I CYP19A1 inhibitors (non-steroidal; anastrazole and letrozole) did not produce obviously cytotoxic effect within 48-hr culture. However, the type II CYP19A1 inhibitor (irreversible; exemestane) exhibited drastically cytotoxic effect within 100 μM treatments. Since SNU1 and SC-M1 are two distinct cell types (SNU1 is non-attached, while SC-M1 is attached to culture dish), we examined long-term exemestane effect using sub-lethal dose (7-days; 25 μM) with flow-cytometry (SNU1) or colony formation (SC-M1) assays. We observed sub-lethal dose exemestane treatment could significantly increase apoptosis of SNU1 cells (sub-GO population from 23% to 73%), while totally suppress SC-M1 colony-formation. In sum our results indicate that CYP19A1 provides a progression confounder in GCa patients, and targeting CYP19A1 with exemestrane provides an effective therapeutic strategy for GCa.

Due to high incidence and poor prognosis, primary tumor removal at early stages of GCa is the only possible curative treatment. However, most patients have unresectable or metastatic disease at diagnosis. In the early 1980s, fluorouracil chemotherapy was evaluated as an active agent for GCa therapy either alone or combined treatment after surgery [32]. However, low response rate (19%-48%) and tolerable toxicity (>50% patients with other gastrointestinal malignancies) make fluorouracil chemotherapy usually serve as reference arm in randomized phase III trials (37). HER2 expression in GCa has received attention as a potential target for therapy with trastuzumab [33], and is standard in the treatment of HER2+ advanced GCa [34]. Unfortunately, there is not better adjuvant therapy for HER2−GCa patients. Although trastuzumab can be applied, relapse in patients is frequent, even when combined with chemotherapies [35, 36]. Cho et al. [37] surveyed the single nucleotide polymorphisms (SNPs) of steroidogenic enzymes for an association with GCa risks, and found inter alia, that CYP19A1 SNPs affect GCa susceptibility; see also: Jin et al., Oncol Lett. 2015 April; 9(4): 1502-1508, Biomarkers for gastric cancer: Progression in early diagnosis and prognosis (Review).

Our disclosure directly links CYP19A1 to patient survival, demonstrating the value of targeting. We disclose that CYP19A1 is a particularly useful targeting site in particular patient populations, e.g., surgery or HER+/− patients. We demonstrate that using human tolerable dose exemestane (25 μM) in GCa cells results in efficient cytotoxicity. Goss et al. (2011)[38] reported that long-term use of exemestane exhibited an excellent breast cancer prevention effect with limited systemic complication. Hence our disclosure indicates that clinical use of exemestane in GCa patients is practical in clinical settings.

Exemestane and 5-FU Synergistically Suppress Gastric Cancer Cell Growth In Vitro

Four groups of treatments (Veh: vehicle; SFU; Exe: Exemestane; and Exe+SFU: exemestane+5-FU) were compared to solvent vehicle treatment. Both 5-FU and Exe suppress human stomach gastric carcinoma (SNU-1) cell growth, and the combination of Exe and 5-FU enhances cell suppression synergistically.

The FIGURE shows cytotoxic effect of the treatments; values are fold of vehicle treatment; p-value (T-Test): Veh vs. 5FU=0.0006, Veh vs. Exemestane=0.0014, Veh vs. Exe+SFU=0.00003.

Exemestane and 5-FU Synergistically Suppress Gastric Cancer Cell Growth In Vivo

We developed our animal human-tumor xenograft protocol to confirm in vivo efficacy and synergy using same four groups we used to demonstrate tumor cell growth inhibition in vitro.

Subcutaneous injection of 10\hat{ }6 human stomach gastric carcinoma (SNU-1) cells/site in athymic nude mice.

After two-wks of injection, start to treat the mice with various drugs (I.P injection; 5-FU: 5 mg/kg/mouse; Exe: 100 mg/kg/mouse) 3 times/wk for consecutive 4-wks.

At each week tumor size are estimated by radiology and body weight are measured.

At trial end-point, blood and tumor tissue is collected for histology, marker, gene expression analysis.

Consistent with our in vitro data, both 5-FU and exemestane suppress human gastric cancer tumor growth in our whole animal xenograft model, and the combination of exemestane and 5-FU enhances tumor growth synergistically.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims. All publications, patents, and patent applications cited herein, including citations therein, are hereby incorporated by reference in their entirety for all purposes

### REFERENCES

1. Thrumurthy S G, Chaudry M A, Chau I and Allum W. Does surgery have a role in managing incurable gastric cancer? Nat Rev Clin Oncol. 2015; 12(11):676-682.
2. Tan P and Yeoh K G. Genetics and Molecular Pathogenesis of Gastric Adenocarcinoma. Gastroenterology. 2015; 149(5):1153-1162 e1153.
3. Bang Y J, et al. Trastuzumab in combination with chemotherapy versus chemotherapy alone for treatment of HER2-positive advanced gastric or gastro-oesophageal junction cancer (ToGA): a phase 3, open-label, randomised controlled trial. Lancet. 2010; 376(9742):687-697.
4. Allum W H, et al. Guidelines for the management of oesophageal and gastric cancer. Gut. 2011; 60(11):1449-1472.
5. Foo M and Leong T. Adjuvant therapy for gastric cancer: current and future directions. World J Gastroenterol. 2014; 20(38):13718-13727.
6. Sun J, Song Y, Wang Z, Chen X, Gao P, Xu Y, Zhou B and Xu H. Clinical significance of palliative gastrectomy on the survival of patients with incurable advanced gastric cancer: a systematic review and meta-analysis. BMC Cancer. 2013; 13:577.
7. Korenaga D, et al. Sex hormone-receptor-negative tumors have a higher proliferative activity than sex hormone-receptor-positive tumors in human adenocarcinomas of the gastrointestinal tract. Surg Today. 1998; 28(10):1007-1014.
8. Duell E J, et al. Menstrual and reproductive factors, exogenous hormone use, and gastric cancer risk in a cohort of women from the European Prospective Investigation Into Cancer and Nutrition. Am J Epidemiol. 2010; 172(12):1384-1393.
9. Cronin-Fenton D P, et al. Reproductive and sex hormonal factors and oesophageal and gastric junction adenocarcinoma: a pooled analysis. Eur J Cancer. 2010; 46(11):2067-2076.
10. Chandanos E and Lagergren J. Oestrogen and the enigmatic male predominance of gastric cancer. Eur J Cancer. 2008; 44(16):2397-2403.
11. Kim J H, Boo Y J, Park J M, Park S S, Kim S J, Kim C S and Mok Y J. Incidence and long-term outcome of young patients with gastric carcinoma according to sex: does hormonal status affect prognosis? Arch Surg. 2008; 143(11):1062-1067; discussion 1067.
12. Wu C W, Chi C W, Chang T J, Lui W Y and P'Eng F K. Sex hormone receptors in gastric cancer. Cancer. 1990; 65(6):1396-1400.
13. Kuru B, Ozaslan C, Yalman K and Camlybel M. Serum progesterone levels in patients with gastric and colorectal cancers. Acta Chir Belg. 2002; 102(2):122-125.
14. Inutsuka S, Kodama Y, Natsuda Y, Kumashiro R and Maekawa T. Serum testosterone level of patients with gastric carcinoma before and after gastrectomy. Cancer. 1986; 58(12):2675-2679.
15. Sah B K, Chen M M, Peng Y B, Feng X J, Yan M, Liu B Y, Fan Q S and Zhu Z G. Does testosterone prevent early postoperative complications after gastrointestinal surgery? World J Gastroenterol. 2009; 15(44):5604-5609.
16. Myant N B. The transport and turnover of the plasma cholesterol. Biochem Soc Symp. 1971; (33):99-121.
17. Caruso M G, Notarnicola M, Cavallini A and Di Leo A. 3-Hydroxy-3-methylglutaryl coenzyme A reductase activity and low-density lipoprotein receptor expression in diffuse-type and intestinal-type human gastric cancer. J Gastroenterol. 2002; 37(7):504-508.
18. Enjoji M, et al. Intracellular mechanisms underlying lipid accumulation (white opaque substance) in gastric epithelial neoplasms: A pilot study of expression profiles of lipid-metabolism-associated genes. J Gastroenterol Hepatol. 2015.
19. Guo E, Chen L, Xie Q, Chen J, Tang Z and Wu Y. Serum HDL-C as a potential biomarker for nodal stages in gastric cancer. Ann Surg Oncol. 2007; 14(9):2528-2534.
20. Li Q, Birkbak N J, Gyorffy B, Szallasi Z and Eklund A C. Jetset: selecting the optimal microarray probe set to represent a gene. BMC Bioinformatics. 2011; 12:474.
21. Gyorffy B, Surowiak P, Budczies J and Lanczky A. Online survival analysis software to assess the prognostic value of biomarkers using transcriptomic data in non-small-cell lung cancer. PLoS One. 2013; 8(12):e82241.
22. Storey S M, et al. Intracellular cholesterol-binding proteins enhance HDL-mediated cholesterol uptake in cultured primary mouse hepatocytes. Am J Physiol Gastrointest Liver Physiol. 2012; 302(8):G824-839.
23. Iacovelli R, Pietrantonio F, Maggi C, de Braud F and Di Bartolomeo M. Combination or single-agent chemotherapy as adjuvant treatment of gastric cancer: A systematic review and meta-analysis of published trials. Crit Rev Oncol Hematol. 2016; 98:24-28.
24. Liu H, et al. The efficacy and toxicity of paclitaxel plus S-1 compared with paclitaxel plus 5-FU for advanced gastric cancer: a PRISMA systematic review and meta-analysis of randomized controlled trials. Medicine (Baltimore). 2014; 93(25):e164.
25. Galdy S, et al. Systemic therapy beyond first-line in advanced gastric cancer: An overview of the main randomized clinical trials. Crit Rev Oncol Hematol. 2015.
26. Lee Y M, Chang W C and Ma W L. Hypothesis: solid tumours behave as systemic metabolic dictators. J Cell Mol Med. 2016.
27. Guillaumond F, et al. Cholesterol uptake disruption, in association with chemotherapy, is a promising combined metabolic therapy for pancreatic adenocarcinoma. Proc Natl Acad Sci USA. 2015; 112(8):2473-2478.
28. Tamura T, Inagawa S, Hisakura K, Enomoto T and Ohkohchi N. Evaluation of serum high-density lipoprotein cholesterol levels as a prognostic factor in gastric cancer patients. J Gastroenterol Hepatol. 2012; 27(10):1635-1640.
29. Dale K M, Coleman C I, Henyan N N, Kluger J and White C M. Statins and cancer risk: a meta-analysis. JAMA. 2006; 295(1):74-80.
30. Shimoyama S. Statins and gastric cancer risk. Hepatogastroenterology. 2011; 58(107-108):1057-1061.
31. Wei W, et al. Ligand Activation of ERRalpha by Cholesterol Mediates Statin and Bisphosphonate Effects. Cell Metab. 2016.
32. Fujii M, Kochi M and Takayama T. Recent advances in chemotherapy for advanced gastric cancer in Japan. Surg Today. 2010; 40(4):295-300.
33. Ieni A, Barresi V, Rigoli L, Caruso R A and Tuccari G. HER2 Status in Premalignant, Early, and Advanced Neoplastic Lesions of the Stomach. Dis Markers. 2015; 2015:234851.
34. Wada R, Hirabayashi K, Ohike N and Morii E. New guidelines for HER2 pathological diagnostics in gastric cancer. Pathol Int. 2016; 66(2):57-62.
35. Chrom P, Stec R and Szczylik C. Second-line Treatment of Advanced Gastric Cancer: Current Options and Future Perspectives. Anticancer Res. 2015; 35(9):4575-4583.
36. Boekhout A H, et al. Trastuzumab. Oncologist. 2011; 16(6):800-810.
37. Cho L Y, et al. Genetic susceptibility factors on genes involved in the steroid hormone biosynthesis pathway and progesterone receptor for gastric cancer risk. PLoS One. 2012; 7(10):e47603.
38. Goss P E, et al. Exemestane for breast-cancer prevention in postmenopausal women. N Engl J Med. 2011; 364(25):2381-2391.
39. Yuan L W, Yamashita H and Seto Y. Glucose metabolism in gastric cancer: The cutting-edge. World J Gastroenterol. 2016; 22(6):2046-2059.
40. Chung I F, Chen C Y, Su S C, Li C Y, Wu K J, Wang H W and Cheng W C. DriverDBv2: a database for human cancer driver gene research. Nucleic Acids Res. 2015; 44(D1):D975-979.
41. Cheng W C, et al. DriverDB: an exome sequencing database for cancer driver gene identification. Nucleic Acids Res. 2014; 42(Database issue):D1048-1054.
42. Chung W M, et al. MicroRNA-21 promotes the ovarian teratocarcinoma PA1 cell line by sustaining cancer stem/progenitor populations in vitro. Stem Cell Res Ther. 2013; 4(4):88.
43. Ma W L, Jeng L B, Lai H C, Liao P Y and Chang C. Androgen receptor enhances cell adhesion and decreases cell migration via modulating beta1-integrin-AKT signaling in hepatocellular carcinoma cells. Cancer Lett. 2014; 351(1):64-71.
44. Chen L, et a. Androgen receptor increases CD133 expression and progenitor-like population that associate with cisplatin resistance in endometrial cancer cell line. Reprod Sci. 2014; 21(3):386-394.

1. A method of treating gastric cancer, comprising administering to a person in need thereof exemestane (6-methylenandrosta-1,4-diene-3,17-dione).

2. The method of claim 1 further comprising the antecedent step of identifying the person as having gastric cancer and/or being in need of exemestane therapy.

3. The method of claim 1 or 2 further comprising the subsequent step of monitoring status of the gastric cancer or a biomarker thereof, such as androgen receptor (AR), progesterone receptor (PR), estrogen receptor 1; ERα (ESR1) and estrogen receptor 2; ERβ (ESR2).

4. The method according to claim 1, 2 or 3, wherein the gastric cancer is HER2-negative.

5. The method according to claim 1, 2, 3, or 4, wherein the person is a gastrectomy patient.

6. The method according to claim 1, 2, 3 or 4, wherein the person is contraindicated for gastrectomy.

7. Use of exemestane (6-methylenandrosta-1,4-diene-3,17-dione) in the manufacture of a medicament for treating gastric cancer.

8. A pharmaceutical composition formulated and/or labeled for treating gastric cancer comprising exemestane (6-methylenandrosta-1,4-diene-3,17-dione).

9. The composition of claim 8 further comprising a different gastric cancer drug, such as 5-FU (fluorouracil) or its analog capecitabine, BCNU (carmustine), methyl-CCNU (semustine), doxorubicin, mitomycin C, cisplatin and taxotere.
